SEQUENCE LISTING (1 sequence)

SEQ ID NO: 1 - protein, 9 aa, Homo sapiens
EILKISEED

(19) United States
(12) Patent Application Publication
(10) Pub. No.: US 2006/0269529 A1
(43) Pub. Date: Nov. 30, 2006

(54) Modified t lymphocytes and uses therefor

(76) Inventors: Thomas M.J. Niederman, Highland Beach, FL (US); Zoher Ghogawala, Greenwich, CT (US); Bos S Carter, Belmont, MA (US); Richard C. Mulligan, Lincoln, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/513,622

(22) PCT Filed: May 7, 2003
(86) PCT No.: PCT/US03/14343

§ 371 Date: Aug. 3, 2006

Related U.S. Application Data
(60) Provisional application No. 60/378,706, filed on May 7, 2002.

(51) Int. Cl. A61K 48/00; C12N 15/867; C12N 5/08

(52) U.S. Cl. 424/93.21; 435/456; 435/372

(57) ABSTRACT

Modified T lymphocytes that express a chimeric T cell receptor reactive with two or more different cell surface angiogenic marker are disclosed. The modified T lymphocytes are useful in methods in controlling angiogenesis and in therapeutic methods in controlling tumor growth.

### RELATED APPLICATION

This application claims the benefit of U.S. Provisional Application No. 60/378,706, filed on May 7, 2002. The entire teachings of this application are incorporated herein by reference.

### GOVERNMENT SUPPORT

The invention was supported, in whole or in part, by grants CA83772-02 and CA73133-01 from the National Cancer Institue. The United States Government has certain rights in the invention.

### BACKGROUND OF THE INVENTION

Angiogenesis is the formation of new blood vessels from existing blood vessels. To initiate the angiogenic process, biochemical signals stimulate protease secretion from, among other cell types, endothelial cells lining the lumen of the vessel. The secreted proteases degrade the basement membrane and the endothelial cell layer protrudes through the hole created in the basement membrane. If the biochemical signals are continuously present, the migrating endothelial cells undergo mitosis and divide. The dividing cells form a sprout through the vessel wall. If the angiogenic stimulus remains, the sprouts merge to form capillary loops which later mature into new blood vessels.

Under normal circumstances of wound healing, fetal and embryonic development and formation of the corpus luteum, endometrium and placenta, the initial angiogenic signals subside and other, secondary, signals predominate to turn off the angiogenic process. However in disease states such as cancer, the local concentration of angiogenic signals never decreases and new blood vessels continuously form.

Angiogenesis, or the recruitment of a new blood supply, is required for the growth of solid tumors (Folkman, J., J. Natl. Cancer Inst., 82:4-6 (1990)). Undesired angiogenesis provides a steady supply of nutrients to the tumor, allowing the tumor to grow and metastasize. Thus, there is intense interest in the development of new therapeutic strategies which target the tumor vasculature.

### SUMMARY OF THE INVENTION

The present invention relates to the development of a novel immune-based antiangiogenic strategy that is based upon the generation of T lymphocytes that possess a killing specificity for cells expressing vascular endothelial growth factor receptors (VEGFRs). As described herein, Applicants have discovered that VEGFR-expressing cells can be targeted using recombinant retroviral vectors that encode a chimeric T cell receptor comprising VEGF sequences linked to intracellular signaling sequences, such as intracellular signaling sequences derived from the ζ chain of the T cell receptor. Applicants have discovered that transduction of cytotoxic T lymphocytes by such vectors results in transduced cells possessing a killing specificity for cells expressing the VEGF2 receptor as measured by in vitro cytotoxicity assays. Additionally, Applicants have discovered that adoptive transfer of the genetically modified cytotoxic T lymphocytes into tumor-bearing mice suppressed or inhibited the growth of a variety of syngeneic murine tumors and human tumor xenografts. As described herein, an increased effect on in vivo tumor growth inhibition can also be obtained when this therapy is combined with the systemic administration of an angiogenesis inhibitor. The utilization of the immune system to target angiogenic markers expressed on tumor vasculature provides a powerful means for controlling tumor growth.

The present invention relates to modified T lymphocytes expressing a chimeric T cell receptor reactive with multiple (two or more) different cell surface angiogenic markers; to retroviral vectors encoding a chimeric T cell receptor reactive with multiple different cell surface angiogenic markers useful for producing the modified T lymphocytes; to packaging cell lines useful for generating the retroviral vectors; to construction of such cell lines; to methods of producing the modified T lymphocytes of the invention using the retroviral vectors; and to methods of using the modified T lymphocytes to target multiple cell surface angiogenesis markers expressed on tumor vasculature, to inhibit angiogenesis and to suppress or inhibit tumor growth. In a preferred embodiment, at least one of the cell surface angiogenic markers that is reactive with the chimeric T cell receptor is a VEGF2 receptor (e.g., kinase insert domain-containing receptor (KDR) or Flk-1).

Modified T lymphocytes of the invention comprise a chimeric T cell receptor reactive with multiple different cell surface angiogenic markers. By “multiple” is meant two or more. Such modified T lymphocytes express the chimeric T cell receptor on their cell surface and possess binding specificity for multiple cell surface angiogenic markers.

Modified T lymphocytes of the invention are produced by transducing lymphocytes with a retroviral vector encoding a chimeric T cell receptor reactive with two or more different cell surface angiogenic markers into T lymphocytes. In a particular embodiment, the transduction protocol comprises (a) pre-activating T lymphocytes using anti-CD28 and anti-CD3 antibodies; (b) co-incubating the activated T lymphocytes on ice with a retroviral vector encoding a chimeric T cell receptor reactive with two or more different cell surface angiogenic markers to produce a suspension of T lymphocytes and retrovirus; and (c) incubating the suspension of T lymphocytes and retrovirus from step (b) at 37° C. in the presence of IL-2 and polybrene.

Retroviral vectors of the present invention that encode a chimeric T cell receptor reactive with two or more different cell surface angiogenic markers comprise (a) a nucleotide sequence coding for a secreted VEGF2 molecule or a binding portion thereof, wherein the VEGF2 molecule or binding portion thereof is capable of binding two or more different cell surface angiogenic markers; (b) an intracellular signal transduction sequence; and (c) a hinge sequence located between the nucleotide of (a) and the intracellular signal transduction sequence of (b). In a particular embodiment, the signal transduction sequence is a nucleotide sequence of the zeta chain of the CD3-T cell receptor complex. In another embodiment, the signal transduction sequence is a nucleotide sequence of the gamma chain of the Fcε receptor I complex (FcεRI) or the Fcγ receptor III complex (FcγRIII). In a further embodiment, the retroviral vector of the invention further comprises a detectable epitope sequence for detecting the chimeric T cell receptor when expressed on transduced T lymphocytes. The nucleic acid sequences comprising the retroviral vectors of the invention are arranged and joined (linked) such that a functional chimeric T cell receptor is encoded.

Chimeric T cell receptors of the invention are reactive with two or more different cell surface angiogenic markers and comprise (a) an extracellular binding domain capable of binding two or more different cell surface angiogenic markers, said binding domain encoded by VEGF2 coding sequences; (b) an intracellular signal-transducing domain; and (c) a hinge region located between the binding domain of a) and the signal-transducing domain of (b). In a particular embodiment, the signal-transducing domain is a domain of the zeta chain of the CD3-T cell receptor complex. In another embodiment, the signal-transducing domain is a domain of the gamma chain of the FcεRI or the FcγRIII. In a further embodiment, the chimeric T cell receptor further comprises a detectable epitope for detecting the chimeric T cell receptor when expressed on transduced T lymphocytes. The domains and regions are arranged and linked such that the resulting chimeric T cell receptor is expressed on the surface of a transduced lymphocyte and is able to participate in signal transduction.

Packaging cell lines for producing a retroviral vector encoding a chimeric T cell receptor reactive with two or more different cell surface angiogenic markers comprise (a) a cell (e.g., a mammalian cell); (b) a first retroviral nucleotide sequence in the cell which comprises a coding sequence for viral gagpol proteins; (c) a second. retroviral nucleotide sequence in the cell which comprises a coding sequence for a heterologous envelope protein; and (d) a third retroviral nucleotide sequence in the cell which comprises a coding sequence for a chimeric T cell receptor reactive with two or more different cell surface angiogenic markers.

Cell lines for producing a retroviral vector encoding a chimeric T cell receptor reactive with two or more different cell surface angiogenic markers are produced by transfecting host cells (e.g., mammalian host cells) with a first plasmid comprising a DNA sequence which encodes viral gagpol proteins; a second plasmid comprising a DNA sequence which encodes a heterologous envelope protein; and a third plasmid comprising a DNA sequence which encodes a chimeric T cell receptor reactive with two or more different cell surface angiogenic markers under conditions appropriate for transfection of the cells.

Methods of the invention for suppressing or inhibiting tumor growth in a patient having a tumor comprise (a) transducing T lymphocytes obtained from the patient with a retroviral vector encoding a chimeric T cell receptor reactive with two or more different cell surface angiogenic markers, thereby producing modified T lymphocytes expressing the chimeric T cell receptor reactive with two or more different cell surface angiogenic markers; and (b) administering to the patient modified T lymphocytes produced in step (a). In a particular embodiment, methods of suppressing or inhibiting tumor growth also comprise administering to the patient an effective amount of IL-2. In a further embodiment, methods of suppressing or inhibiting tumor growth further comprise administering to the patient an angiogenesis inhibitor.

### BRIEF DESCRIPTION OF THE DRAWINGS

FIG. 1A shows schematic diagrams depicting the structure of retroviral vectors encoding a VEGF chimeric T cell receptor, a truncated form of the VEGF chimeric T cell receptor or a MR-1 single chain monoclonal antibody chimeric T cell receptor. Abbreviations: VEGF-cTcR, vascular endothelial cell growth factor chimeric T cell receptor; MR-1-cTcT, MR-1 single chain monoclonal antibody chimeric T cell receptor; SD, splice donor; SA, splice acceptor; CMV IE, cytomegalovirus immediate early promoter; myc, human c-Myc epitope; CD8, CD8α hinge region; TCR, ζ chain of the T cell receptor; LTR, long terminal repeat.

FIG. 1B shows plots depicting the results of fluorescence-activated cell sorter (FACS) analysis of untransduced cytotoxic T lymphocytes (CTLs) (dashed line) or CTLs transduced with VEGF-cTcR or MR-1-cTcR (solid line). Splenocytes transduced with VEGF-cTcR were also incubated with anti-CD8-FITC antibody (solid line) or an anti-rat IgG-FITC negative control antibody (dashed line).

FIG. 1C shows plots depicting the results of FACS analysis of VEGF-cTcR clone 2 and MR-1-cTcR clone 8 cells.

FIGS. 2A to 2B show plots depicting the results of an assessment of binding of Flk-1 to cTcR-expressing cells. FIG. 2A shows plots of the results of FACS analysis demonstrating the binding of soluble Flk-Fc only to HeLa cells expressing VEGF-cTcR. CM, conditional medium. FIG. 2B is a plot of the results of binding of soluble human KDR-Fc to a CTL clone expressing VEGF-cTcR (VEGF-cTcR clone 2). Cells were incubated with increasing concentrations of soluble KDR-Fc, washed, incubated with anti-human IgG-FITC and subject to FACS analysis The data are plotted on a logarithmic scale. The insert is a plot of the data on a linear scale to demonstrate saturable binding.

FIGS. 3A to 3C show plots depicting results showing that VEGF-cTcR T-cells specifically lyse cells expressing Flk-1. In FIG. 3A, primary VEGF-cTcR CTLs (squares) or MR-1-cTcR CTLs (circles) were incubated with either B16.F10 cells that either expressed (▪ and ●, respectively) or did not express (□ and ◯, respectively) Flk-1 at varying effector-to-target ratios, and cell lysis was determined using a standard Cr⁵¹ release assay. In FIG. 3B, primary VEGF-cTcR CTLs (▪), VEGF-cTcR del Z CTLs (□) or MR-1-cTcR CTLs (●) were incubated with adherent murine islet endothelial (MILE) cells, and lysis was determined using a standard dehydrogenase (LDH) release assay. In FIG. 3C, MILE cells were preincubated with no antibodies, anti-Flk-1 antibodies or isotype-control (IC) antibodies before incubation with CTLs expressing VEGF-cTcR (filled bars) or VEGF-cTcR del Z (open bars) in a 5 hour cytotoxicity assay at an effector-to-target ratio of 15:1. Each data point reflects the mean of six independent determinations.

FIGS. 4A to 4D show plots depicting the results of adoptive inmmunotherapy using genetically modified CTLs. In FIG. 4A, CT26 cells (5×10⁵, n=6 per group) were implanted subcutaneously on BALB/c mice. In FIG. 4B, B16.F10 cells (7×10⁵, n=4 per group) were implanted on C57BL/6 mice. In FIG. 4C, B16.F10 cells (5×10⁵, n=4 per group) were implanted on C57BL/6 nude mice. In FIG. 4D, LS174T cells (1×10⁶, n=7 per group) were implanted on C57BL/6 nude mice. On the days indicated with an arrowhead, mice were treated with 5×10⁶ to 9×10⁶ VEGF-cTcR CTLs (□), MR-1-cTcR CTLs ( [symbol] ), phosphate buffered saline (PBS) (◯), PBS with no exogenous IL-2 (▾) or VEGF-cTcR del Z (X). Daily intraperitoneal injections of IL-2 started on the first day of CTL infusion (except for the group indicated with the inverted triangle in FIG. 4B). Tumor volume was calculated using the formula width²×length×0.52, and standard error of the mean (SEM) is indicated with error bars. The ratio of the tumor volumes of the VEGF-cTcR CTL-treated mice to the PBS control mice (T/C) was determined for the last time point.

FIGS. 5A to 5C show plots depicting the results of the effects of the combined treatment of genetically modified CTLs and TNP-470 on tumor growth. In FIG. 5A, CT26 adenocarcinoma cells (5×10⁵, n=4 per group) were implanted subcutaneously on BALB/c mice. In FIG. 5B, B16.F10 melanoma cells (7×10⁵, n =4 per group) were implanted on C57BL/6 mice. In FIG. 5C, T241 fibrosarcoma cells (5×10⁵, n=3 per group) were implanted on C57BL/6 mice. On the days indicated with an arrowhead, mice were treated with 5×10⁶ to 10×10⁶ VEGF-cTcR CTLs (□), VEGF-cTcR CTLs+TNP-470 (▪), MR-1-cTcR CTLs+TNP-470 (▴), PBS+TNP-470 (●) or PBS (◯). Mice were treated with TNP-470 every other day and IL-2 every day starting on the first day of CTL therapy. Tumor measurements and analyses were as described for FIGS. 4A-4D. T/C, ratio of the tumor volumes of the VEGF-cTcR CTL-treated mice to the PBS control mice.

### DETAILED DESCRIPTION OF THE INVENTION

The present invention relates to modified T lymphocytes expressing a chimeric T cell receptor reactive with two or more different cell surface angiogenic markers, to viral vectors encoding a chimeric T cell receptor reactive with multiple different cell surface angiogenic markers useful for producing the modified T lymphocytes, to packaging cell lines useful for generating the viral-vectors, to construction of such cell lines, to methods of producing the modified T lymphocytes of the invention using the viral vectors and to methods of using the modified T lymphocytes to suppress or inhibit tumor growth. By “angiogenic marker” is meant a growth factor receptor or other binding specificity that controls angiogenesis, a multi-part process that involves cooperative interactions between multiple growth factor receptor/ligand pairs. By “control” is meant the ability to affect the rate and extent to which a process occurs. In a preferred embodiment, at least one of the cell surface angiogenic markers that is reactive with the chimeric T cell receptor is a VEGF2 receptor (e.g., KDR or Flk-1).

Modified T lymphocytes of the invention comprise a chimeric T cell receptor reactive with multiple different cell surface angiogenic markers. Such modified T lymphocytes express the chimeric T cell receptor on their cell surface and possess binding specificity for multiple cell surface angiogenic markers. Accordingly, the modified T lymphocytes of the invention are able to target multiple different cell surface angiogenic markers expressed on tumor vasculature. Targeting and blocking of or interfering with multiple cell surface angiogenic markers provides a more powerful an effective means to prevent the formation of new blood vessels and destroy existing tumor vasculature because interactions between multiple growth factor receptor/ligand pairs specific for tumor neovasculature can be blocked.

Modified T lymphocytes of the invention are produced by transducing T lymphocytes with a viral vector encoding a chimeric T cell receptor reactive with two or more different cell surface angiogenic markers into T lymphocytes. In a particular embodiment, the transduction protocol comprises (a) pre-activating T lymphocytes using anti-CD28 and anti-CD3 antibodies; (b) co-incubating the activated T lymphocytes on ice with a viral vector encoding a chimeric T cell receptor reactive with two or more different cell surface angiogenic markers to produce a suspension of T lymphocytes and virus; and (c) incubating the suspension of T lymphocytes and virus from step (b) at 37° C. in the presence of IL-2 and polybrene. Transduction can also be carried out by other methods known in the art, such as, for example, microinjection, electroporation, retroviral transduction or transfection using DEAE-dextran, lipofection, calcium phosphate, particle bombardment mediated gene transfer (see, e.g., Sambrook et al., Molecular Cloning. A Laboratory Manual (Plainview, N.Y.: Cold Spring Harbor Press) (1989)).

In a particular embodiment, the viral vector is a retroviral vector. In another particular embodiment, the viral vector is a lentiviral vector. Other viral vectors include adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomregalovirus) and poxvirus (e.g., vaccinia, fowlpox and canarypox). Still other viruses include Norwalk virus, togavirus, flavivirus, reoviruses, papovavirus, hepadnavirus and hepatitis virus, for example. Examples of retroviruses include avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, 3rd Edition, B. N. Fields et al., eds. (Philadelphia, Pa.: Lippincott-Raven Publishers) (1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus and Rous sarcoma virus.

Viral vectors encoding a chimeric T cell receptor reactive with two or more different cell surface angiogenic markers comprise (a) a nucleotide sequence coding for a secreted VEGF2 molecule or a binding portion thereof, wherein the VEGF2 molecule or binding portion thereof is capable of binding two or more different cell surface angiogenic markers; (b) an intracellular signal transduction sequence; and (c) a hinge sequence located between the nucleotide of (a) and the intracellular signal transduction sequence of (b). In a particular embodiment, the viral vector further comprises a detectable epitope sequence for detecting the chimeric T cell receptor when expressed on transduced T lymphocytes.

The intracellular signal transduction sequence (or domain), also referred to herein as an “intracellular signaling sequence” (or domain), is important for expression and signaling. In a particular embodiment, the signal transduction sequence is a nucleotide sequence of the zeta chain of the CD3-T cell receptor complex. In another embodiment, the signal transduction sequence is a nucleotide sequence of the gamma chain of the Fcε receptor I complex (FcεRI) or the Fcγ receptor III complex (FcγRIII.)

The hinge region (sequence) is a flexible domain that joins the extracellular and intracellular domains of a chimeric T cell receptor. The hinge region of the chimeric T cell receptor provides flexibility at the juxtamembrane surface and improves cell surface expression of the chimeric T cell receptor and signaling. In a particular embodiment, the hinge sequence encodes the CD8α hinge region. In another embodiment, the hinge sequence encodes the transmembrane domain of CD28.

The viral vectors of the invention are constructed using conventional methods known in the art (see, e.g., Ausubel et al., Current Protocols in Molecular Biology (New York: John Wiley & Sons, Inc.) (1998); Sambrook et al., Molecular Cloning: A Laboratory Manual, 2nd edition (New York: Cold Spring Harbor University Press (1989)). The nucleic acid sequences of the viral vectors of the invention are arranged and joined such that a functional chimeric T cell receptor is encoded.

Chimeric T cell receptors of the invention are reactive with two or more different cell surface angiogenic markers and comprise (a) an extracellular binding domain capable of binding two or more different cell surface angiogenic markers, said binding domain encoded by VEGF2 coding sequences; (b) an intracellular signal-transducing domain; and (c) a hinge region located between the binding domain of a) and the signal-transducing domain of (b). In a particular embodiment, the signal-transducing domain is a domain of the zeta chain of the CD3-T cell receptor complex. In another embodiment, the signal-transducing domain is a domain of the gamma chain of the FcεRI or the FcγRIII. In a further embodiment, the chimeric T cell receptor further comprises a detectable epitope for detecting the chimeric T cell receptor when expressed on transduced T lymphocytes. The domains and regions are arranged and linked such that the resulting chimeric T cell receptor is expressed on the surface of a transduced lymphocyte and is able to participate in signal transduction.

By “VEGF2 coding sequences” is meant nucleotide sequences encoding a secreted VEGF molecule or a binding portion thereof. Such sequences include the VEGF-165 coding sequence and fragments encoding binding portions thereof. The nucleotide sequences may correspond to natural sequences or any sequences which encode the protein in its natural amino acid sequence or a mutein characterized by minor modifications to the amino acid sequence such that the mutant protein is substantially similar in amino acid sequence and/or 3D structure and possesses a similar binding ability relative to the native protein.

Nucleotide sequences can be isolated from nature, modified from native sequences or manufactured de novo, as described in, for example, Ausubel et al., Current Protocols in Molecular Biology, John Wiley & Sons, New York (1998); and Sambrook et al., Molecular Cloning: A Laboratory Manual, 2nd edition, Cold Spring Harbor University Press, New York. (1989). Nucleotide sequences can be isolated and fused together by methods known in the art, such as exploiting and manufacturing compatible cloning or restriction sites.

Packaging cell lines for producing a viral vector of the invention encoding a chimeric T cell receptor reactive with two or more different cell surface angiogenic markers comprise (a) a mammalian cell; (b) a first viral nucleotide sequence in the cell which comprises a coding sequence for viral gagpol proteins; (c) a second viral nucleotide sequence in the cell which comprises a coding sequence for a heterologous envelope protein; and (d) a third viral nucleotide sequence in the cell which comprises a coding sequence for a chimeric T cell receptor reactive with two or more different cell surface angiogenic markers.

Cell lines for producing a viral vector encoding a chimeric T cell receptor reactive with two or more different cell surface angiogenic markers are produced by transfecting host cells (e.g., mammalian host cells) with a first plasmid comprising a DNA sequence which encodes viral gagpol proteins; a second plasmid comprising a DNA sequence which encodes a heterologous envelope protein; and a third plasmid comprising a DNA sequence which encodes a chimeric T cell receptor reactive with two or more different cell surface angiogenic markers under conditions appropriate for transfection of the cells.

A plasmid comprising DNA sequences which encode virus gagpol proteins is also referred to a packaging construct. This plasmid includes a promoter which drives the expression of the gagpol proteins, such as the human cytomegalovirus (hCMV) immediate early promoter. The term “promoter”, as used herein, refers to a sequence of DNA, usually upstream (5′) of the coding region of a structural gene, which controls the expression of the coding region by providing recognition and binding sites for RNA polymerase and other factors which may be required for initiation of transcription. Other suitable promoters are well known and readily available in the art (see, e.g., Ausubel et al., Current protocols in Molecular Biology (New York: John Wiley & Sons, Inc.) (1998); Sambrook et al., Molecular Cloning: A Laboratory Manual, 2nd edition (New York: Cold Spring Harbor University Press (1989); and U.S. Pat. No. 5,681,735).

As used herein, a heterologous envelope protein permits pseudotyping of particles generated by the packaging construct and includes the G glycoprotein of vesicular stomatitis virus (VSV G) and the amphotropic envelope of the Moloney leukemia virus (MLV). A plasmid comprising a DNA sequence which encodes a heterologous envelope protein is also referred to as an envelope coding plasmid.

The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mamnmals). Examples of mammalian species include humans and other primates (e.g., monkeys, chimpanzees), rodents (e.g., rats, mice, guinea pigs) and ruminents (e.g., cows, pigs, horses).

Examples of mammalian cells include human (such as HeLa cells, 293T cells, NIH 3T3 cells), bovine, ovine, porcine, murine (such as embryonic stem cells), rabbit and monkey (such as COS1 cells) cells. The cell may be a non-dividing cell (including hepatocytes, myofibers, hematopoietic stem cells, neurons) or a dividing cell. The cell may be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions).

Typically, cells isolated from a specific tissue (such as epithelium, fibroblast or hematopoietic cells) are categorized as a “cell-type.” The cells can be obtained commercially or from a depository or obtained directly from an animal, such as by biopsy. Alternatively, the cell need not be isolated at all from the animal where, for example, it is desirable to deliver the virus to the animal in gene therapy.

Virus stocks consisting of viral vector particles of the present invention are produced by maintaining the transfected host cells under conditions suitable for virus production (e.g., in an appropriate growth media and for an appropriate period of time). Such conditions, which are not critical to the invention, are generally known in the art. See, e.g., Sambrook et al., Molecular Cloiziitg: A Laboratory Manual, Second Edition, Cold Spring Harbor University Press, New York (1989); Ausubel et al., Current Protocols in Molecular Biology, John Wiley & Sons, New York (1998); U.S. Pat. No. 5,449,614; and U.S. Pat. No. 5,460,959, the teachings of which are incorporated herein by reference.

The packaging cell lines and viral particles of the present invention can be used to produce modified T lymphocytes expressing a chimeric T cell receptor reactive with multiple different cell surface angiogenic markers, in accordance with the methods described herein. Lymphocytes to be transduced using the packaging cell lines and viral particles of the invention can be obtained commercially or from a depository or obtained directly from a mammal, such as by biopsy. Lymphocytes can be obtained from a mammal to whom they will be returned or from another/different mammal of the same or different species.

Modified T lymphocytes of the invention can be used to target multiple different cell surface angiogenic markers expressed on tumor vasculature, to block/inhibit angiogenesis and to suppress/inhibit tumor growth. Current methods of administering such modified T lymphocytes involve adoptive immunotherapy or cell-transfer therapy. These methods allow the return of the modified T lymphocytes to the blood stream. See, e.g., Rosenberg, S. A., Scientific American, 262:62-69 (1990); and Rosenberg, S. A. et al., The N. Engl. J. Med., 323(9):570-578 (1990).

Modified T lymphocytes of the present invention can also be further engineered to secrete additional gene products possessing antiangiogenic and/or other antitumor activities.

Modified T lymphocytes of the present invention can be administered in the form of a pharmaceutical composition with suitable pharmaceutically acceptable excipients. Such compositions can be administered to any animal which may experience the beneficial effects of the modified T lymphocytes of the present invention, including humans.

In a particular embodiment, the invention provides methods of suppressing or inhibiting tumor growth in a patient having a tumor comprising (a) transducing T lymphocytes obtained from the patient with a viral vector encoding a chimeric T cell receptor reactive with two or more different cell surface angiogenic markers, thereby producing modified T lymphocytes expressing the chimeric T cell receptor reactive with two or more different cell surface angiogenic markers; and (b) administering to the patient modified T lymphocytes produced in step (a). In a particular embodiment, methods of suppressing or inhibiting tumor growth also comprise administering to the patient an effective amount of IL-2. In a further embodiment, methods of suppressing or inhibiting tumor growth fuirther comprise administering to the patient an angiogenesis inhibitor. Angiogenesis inhibitors include, for example, fumagillin analogs, endostatin, angiostatin, soluble VEGF receptors, thalidomide and TNFα.

Modified T lymphocytes of the invention can be administered to a mammal, preferably a human, in a therapeutically effective amount. The dosages or number of T lymphocytes administered to a mammal, including frequency of administration, will vary depending upon a variety of factors, including mode and route of administration; size, age, sex, health, body weight and diet of the recipient mammal; nature and extent of symptoms of the disease or disorder being treated; kind of concurrent treatment, frequency of treatment, and the effect desired. The dosing regimes or ranges of lymphocytes used in conventional tumor infiltrating lymphocyte (TIL) therapy (Rosenberg, S. A. et al., J. Natl. Canc. Inst., 86:1159-1166 (1994) can be used as general guidelines for the doses or number of modified T lymphocytes to be administered to a patient in need of such treatment.

Methods for administering (introducing) modified T lymphocytes of the invention to a mammal are generally known to those practiced in the art. For example, modes of administration include parenteral, injection, mucosal, systemic, implant, intraperitoneal, intralesionally, intravenous including infusion and/or bolus injection, subcutaneous, topical, epidural, etc.

An effective amount of IL-2 is that amount, or dose, administered to a mammal that is required to achieve therapeutic effect when administered in conjunction with the modified T lymphocytes of the invention. The effective amount of IL-2 is not necessarily that amount required to achieve therapeutic effect when IL-2 is administered alone. IL-2 is generally provided at a dosage range of 1,200 IU/kg to 1,200,000 IU/kg.

Angiogenesis inhibitors can be administered to a mammal, preferably a human, in a therapeutically effective amount. The dosage or amount of angiogenesis inhibitor administered to a mammal, including frequency of administration, will vary depending upon a variety of factors, including the specific angiogenesis inhibitor administered; the mode and route of administration; size, age, sex, health, body weight and diet of the recipient mammal; nature and extent of symptoms of the disease or disorder being treated; kind of concurrent treatment, frequency of treatment, and the effect desired.

Tumor growth is dependent on angiogenesis involving the proliferation and migration of endothelial cells in response to growth factors, including vascular endothelial growth factor (VEGF) (Hanahan, D. et al., Cell, 86:353-364 (1996)). Many therapeutic strategies examined to date involve the use of either small molecules (Vajkoczy, P. et al., Neoplasia, 1:31-41 (1999); Kusaka, M. et al., Br. J. Cancer, 69:212-216 (1994); Laird, A. D. et al., Cancer Res., 60:4152-4160 (2000); Mendel, D. B. et al., Clin. Cancer Res., 6:4848-4858 (2000); and Strawn, L. M. et al., Cancer Res., 56:3540-3545 (1996)) or soluble forms of endothelial growth factor receptors (Kuo, C. J. et al., Proc. Natl. Acad. Sci. USA, 98:4605-4610 (2001); Goldman, C. K. et al., Proc. Natl. Acad. Sci. USA, 95:8795-8800 (1998); and Aiello, L. P. et al., Proc. Natl. Acad. Sci. USA, 92:10457-10461 (1995)) to interfere with the further development of tumor vasculature. In addition to these cytostatic strategies (Brower, V., Nat. Biotechnol., 17:963-968 (1999)), other approaches aimed at the direct destruction of the tumor vasculature have recently been described that make use of toxins (Backer, M. V. et al., J. Control Release, 74:349-355 (2001); Olson, T. A. et al., Int. J. Cancer, 73:865-870 (1997); Ramakrishnan S. et al., Cancer Res., 56:1324-1330 (1996); and Ramakrishnan S. et al., Methods Mol. Biol., 166:219-234 (2001)) or thrombotic agents (Burrows, F. J. et al., Pharmacol. Ther., 64:155-174 (1994); and Burrows, F. J. et al., Proc. Natl. Acad. Sci. USA, 90:8996-9000 (1993)) that have been conjugated to endothelial cell receptor ligands or antibodies. These latter cytotoxic strategies, in principle, could provide for potent and long-lasting inhibition of tumor growth, because they are potentially able to both prevent the formation of new vessels and destroy existing tumor vasculature.

The present invention expands the power of such cytotoxic strategies. The present invention relates to the development of a cell-based therapy aimed at both the immune-mediated destruction of tumor vasculature and the targeted delivery of biologically active gene products to sites of tumor and its associated vasculature. As described herein, chimeric T cell receptor technology (Altensclmiidt, U. et al., J. Immunol., 159:5509-5515 (1997); Romeo, C. et al., Cell, 64:1037-1046 (1991); and Eshhar, Z. et al., Proc. Natl. Acad. Sci. USA, 90:720-724 (1993)) was used in conjunction with gene transfer to generate cytotoxic T cells capable of recognizing and killing cells which express vascular endothelial growth factor 2 receptor (VEGFR2), a receptor involved in the growth of tumor vessels, in an MHC-independent fashion. Specific recombinant retroviruses encoding such a chimeric receptor comprising VEGF-coding sequences linked to a signaling sequence, such as the signaling ζ chain of the T cell receptor (TCR) or the gamma chain of the FcεRI or the FcγRIII, were constructed as described herein. Primary T lymphocytes transduced by the vectors were shown to efficiently and specifically kill VEGFR2-bearing cells in vitro. Additionally, adoptive transfer of the genetically modified T lymphocytes into tumor-bearing mice suppressed or inhibited the growth of a variety of syngeneic murine tumors and human tumor xenografts.

The studies reported herein indicate that the adoptive transfer of primary T cells possessing a killing specificity for VEGFR2 results in the potent inhibition of tumor growth. Critical to the studies was the use of chimeric T cell receptor technology, which makes it possible to generate cytotoxic T cells possessing an MHC-independent killing specificity for virtually any antigen (Romeo, C. et al., Cell, 64:1037-1046 (1991); Eshhar, Z. et al., Proc. Natl. Acad. Sci. USA, 90:720-724 (1993); and Moritz, D. et al., Proc. Natl. Acad. Sci. USA, 91:4318-4322 (1994)). Although in previous studies, chimeric T cell receptors have been generated against either tumor specific or tumor associated antigens (Altenschmidt, U. et al., J. Immunol., 159:5509-5515 (1997); Eshhar, Z. et al., Proc. Natl. Acad. Sci. USA, 90:720-724 (1993); Moritz, D. et al., Proc. Natl. Acad. Sci. USA, 91:4318-4322 (1994); Hwu, P. et al., Cancer Res., 55:3369-3373 (1995); Darcy, P. K. et al., J. Immunol., 164:3705-3712 (2000); Hekele, A. et al., Int. J. Cancer, 68:232-238 (1996); Brocker, T. et al, Adv. Immunol., 68:257-269 (1998); and McGuinness, R. P. et al., Hum. Gene Ther., 10:165-173 (1999)), the studies herein have made use of the technology to direct an immune response to what is essentially a self-antigen expressed primarily, although not exclusively, on proliferating endothelial cells. Although the generation of an apparently conceptually similar chimeric receptor based on a single chain antibody to flk-1 has been reported (Kershaw, M. H. et al, Hum. Gene Ther., 11:2445-2452 (2000)), the chimeric T cell receptor of the present invention is reactive with multiple (two or more) different cell surface angiogenic markers. Accordingly, the chimeric T cell receptor of the invention is able to target multiple different cell surface angiogenic markers expressed on tumor vasculature. Targeting and blocking of or interfering with multiple cell surface angiogenic markers provides a more powerful and effective means to prevent the formation of new blood vessels and destroy existing tumor vasculature because interactions between multiple growth factor-receptor pairs specific for tumor neovasculature can be blocked.

On the basis of the in vitro cytotoxicity observed with VEGF-cTcR-bearing T cells and the dependence of the therapeutic efficacy of the genetically modified cells on a chimeric T cell receptor capable of signaling, it is likely that the anti-tumor activity is caused by the cytotoxic activity of the cells. However, additional experiments can be conducted to examine the role of the release of cytotoxic or inhibitory cytokines in the milieu of the tumor neovascular network or the role of interference with the incorporation of new endothelial cells into the vascular network in anti-tumor activity. Although efforts to directly observe the destruction of tumor vasculature through measurements of microvessel density have been unsuccessful, recent studies suggest that measurements of microvessel density may not be informative of successful angiogenic blockade, because successful inhibition of tumor angiogensis can lead to a simultaneous decrease in tumor mass and tumor microvessels that results in no apparent change in microvessel density. Additional studies can be conducted to further examine the mechanistic bases underlying the inhibition of tumor growth observed.

The studies presented herein represent an important proof-of-principle for the development of immune-based antiangiogenic therapies. A variety of issues regarding such a therapy can be further examined. First, from the standpoint of toxicity, the experiments herein indicated that animals treated with T cells expressing the VEGF-cTcR exhibited no obvious toxicity with respect to changes in weight, general appearance, or behavior, despite repeated CTL infusions. Flk-1, the target of the chimeric receptor, is known to be expressed at some level in several normal tissues, including the retina (Cheever, M. A. et al., Immunol. Rev., 157:177-194 (1997)), kidney (Feng, D. et al., J. Histochem. Cytochem., 48:545-556 (2000)) and pancreas (Christofori, G. et al., Mol. Endocrinol., 9:1760-1770 (1995)). Additional detailed studies of those specific tissues/organs can be conducted to define the levels of Flk-1 required for efficient recognition by chimeric receptor-bearing cells and to evaluate more fully the potential toxicities of the therapy as described herein. In this regard, cTcRs which target gene products that are more specifically expressed on tumor neovasculature (St. Croix, B. et al., Science, 289:1197-1202 (2000)) can be generated.

To improve the overall therapeutic efficacy of T cells directed toward the tumor vasculature, the use of chimeric receptors capable of providing signals for co-stimulation has been explored (Beecham, E. J. et al., J. Immunother., 23:631-642 (2000); Finney, H. M. et al., J. Immunol., 161:2791-2797 (1998); and Hombach, A. et al., Cancer Res., 61:1976-1982 (2001)) in an effort to generate cells capable of long term persistence in vivo and evaluating combination therapies involving the administration of other antiangiogenic and cytotoxic agents in conjunction with targeted CTL therapy. In addition, because the form of cytotoxic T cell therapy described herein provides a means of targeting genetically modified cells to sites of tumor and tumor vasculature, the use of cTcR bearing cells further engineered to secrete additional gene products possessing antiangiogenic and/or other anti-tumor activities may fuirther enhance the anti-tumor activity of these cells.

The present invention will now be illustrated by the following example, which is not to be considered limiting in any way.

### EXAMPLE

The following materials and methods were used in this example.

### Mice

Mice were purchased from Taconic Farms, and all animal work was conducted at the Harvard Institutes of Medicine Animal Facility in accordance with institutional guidelines.

### Cell Lines

HeLa, B16.F10 and LS174T cells were obtained from the American Type Culture Collection (ATCD). T241 (murine fibrosarcoma) and murine islet endothelial (MILE) cells (syngeneic with C57BL/6) cells were provided by Judah Folkman (Children's Hosptial, Boston). MILE cells were grown in DMEM supplemented with 10% inactivated fetal serum, 10% Nu serum IV and 10 ng/ml basic fibroblast growth factor (Becton-Dickinson) in a 10% CO₂ incubator. The CL96 cytotoxic T-cell line was provided by Uwe Altenschindt (Marcucci, F. et al., Nature, 291:79-81 (1981)). CTLs were maintained in T cell growth media (TCGM): RPMI 1640 supplemented with 10% fetal calf serum, 1 mM pyruvate, 100 units/ml penicillin, 100 μg/ml streptomycin, 2 mM glutamine, 20 mM HEPES, 0.1 mM nonessential amino acids, 64 μM 2-mercaptoethanol and 2 ng/ml human recombinant IL-2 (Sigma).

### Retroviral Vector Construction

Murine VEGF-164 cDNA was provided by Bruce Spiegelman (DFCI, Boston) (Claffey, K. P., J. Biol. Chem., 267:16317-16322 (1992)), murine CD8α cDNA by Dan Littman (New York University, New York) and murine T-cell receptor (TCR) ζ chain cDNA by Bernd Groner (Moritz, D. et al, Proc. Natl. Acad. Sci. USA, 91:4318-4322 (1994)). CMMP-VEGF-cTcR was created by fusing the murine VEFG-165 coding sequence to a human c-Myc epitope (EILKISEED; SEQ ID NO:1), the hinge region of murine CD8α, and the murine TCR ζ chain (Moritz, D. et al., Proc. Natl. Acad. Sci. USA, 91:4318-4322 (1994)) using standard molecular biology techniques. CMMP-VEGF-cTcR del Z was generated by using a synthetic double-stranded oligonucleotide bearing BamHI and BspEI sticky ends to replace the 0.2 kB BamHI-BspEI fragment of CMMP-VEGF-cTcR. This oligonucleotide substituted a CAG codon (Q) for the first intracytoplasnic TAC codon (Y) and introduced a TAA stop codon 15 amino acids downstream, thus eliminating the C-terminal 100 amino acids. Murine Flk-1 cDNA was provided by Ihor Lemischska (Princeton University, Princeton), and the entire Flk-1 coding sequence was inserted between the NcoI and BamHI sites in the SFG retroviral vector (SFG-Flk-1) (Matthews, W. et al., Proc. Natl. Acad. Sci. USA, 88:9026-9030 (1991)). Soluble Flk-Fc was created by cloning the Fc portion of murine IgG₂ₐ between the BsaBI and BamHI sites of SFG-Flk-1, thereby replacing the transmembrane and intracellular domain of Flk-1. The MR-1 gene encodes a single-chain monoclonal antibody directed against a mutant EGFRvIII receptor (Wikstrand, C. J. et al., Cancer Res., 55:3140-3148 (1995)) and was assembled (Stemmer, W. P. et al., Gene, 164:49-53 (1995)) by using synthetic oligonucleotides corresponding to the published cDNA sequence. MR-1 sequences were cloned between the XbaI and BglII sites into CMMP-VEGF-cTcR, replacing VEGF, to generate CMMP-MR-1cTcR.

### Retrovirus Production

293T cells were grown to 50-60% confluence in 150 mm plates and underwent a tripartite transfection with the following plasmids by using a standard calcium phosphate protocol (Soneoka, Y. et al., Nucleic Acids Res., 23:628-633 (1995)): pMD-gag-pol (Ory, D. S. et al., Proc. Natl. Acad. Sci. USA, 93:11400-11406 (1996)) (35 μg), pMD-G (Ory, D. S. et al., Proc. Natl. Acad. Sci. USA, 93:11400-11406 (1996)) (35 μg), and either CMMP-VEGF-cTcR (40 μg) or CMMP-MR-1-cTcR (40 μg). Viral supernatant was removed at 28 hours after DNA addition, passed through a 0.45 μm filter (Nalgene, Rochester, N.Y.) and refrigerated. Concentrated viral stocks were prepared by centrifugation of viral supernatant in an SW28 rotor at 25,000×g, 4° C., for 1.5 hours. The supernatant was decanted and 250 μl of TNE (50 mM Tris, pH 7.8, 130 mM NaCl and 1 mM EDTA, pH 8.0) was added. After sitting overnight at 4° C., virus was resuspended and stored at −80° C. The titers of unconcentrated and concentrated viral stocks on NIH 3T2 cells were approximately 4×10⁶ and 3×10⁸ infectious particles per milliliter, respectively, as determined by FACS analysis.

### Retroviral Transduction of Cell Lines

HeLa cells were transduced with CMMP-VEGF-cTcR retrovirus. After three days of culture, populations of HeLa cells expressing VEGF-cTcR were FACS sorted using anti-Myc antibody treatment followed by anti-mouse IgG₂ₐ-PE (phycoerythrin). CL96 cells were transduced with either CMMP-VEGF-cTcR or CMMP-MR-1-cTcR as described below for primary splenocytes. Three days after transduction, single-cell clones were established by limiting dilution in 96-well plates.

### Splenocyte Harvest and Retroviral Transduction of CTLs

Spleens were harvested and crushed through a 70 μm nylon filter. Following red-cell lysis, CD8+ splenocytes were obtained using negative selection columns (Cytovax Biotechnologies, Edmonton, AB, Canada). CTLs were seeded in 6-well plates pre-coated with 2 μg of anti-mouse CD29 and 2 μg anti-mouse CD3e antibodies (Pharmingen) per well. Three days later, the CTLs were harvested and pooled, and 1×10⁶ cells were transferred to conical tubes and centrifuged at 1,000 rpm. Supernatant was removed, leaving 100 μl to cover the cells. Fifty microliters of concentrated viral stock and 250 μl PBS was added, and cells were resuspended and placed on ice for 3 hours. Cells were then transferred to 12-well plates (pre-coated as described above), and 400 μl of TCGM containing 4 ng/ml IL-2 and 16 μg/ml polybrene was added to each well. After 6 hours at 37° C., 1 ml of TCGM was added to each well. After an additional 12 hours, the cells were washed with PBS and replated in pre-coated dishes. On days 3 and 5 post-transduction, cells were re-fed and split into uncoated 6-well dishes.

### Southern Blot Analysis

Five days after retroviral transduction, CD8+ splenocytes were harvested. Genomic DNA was prepared from viable cells and digested overnight with XbaI and BglII. Filters were incubated with the ³²P-labeled ApaLI-AflIII 453-bp fragment of CMMP-VEGF-cTcR, washed and exposed to film.

### FACS Analysis

High-titer retrovirus encoding the gene for a soluble form of Flk-1, Flk-Fc, were used to infect 293T cells. Conditioned medium from these transduced cells was collected and subjected to Western blot analysis by using either antibody directed against Flk-1 or ¹²⁵I-labeled protein A to confirm the presence of both the Flk-1 and Fc portions of the fusion protein. For FACS analysis of HeLa-VEGF-cTcR cells, 2 ml of conditioned medium from these transduced cells or control media from untransduced 293T cells was used as the primary reagent. The cells were then washed and incubated with anti-mouse IgG₂ₐ-PE (Chemicon) and analyzed. For the VEGF-cTcR/KDR-Fc binding study, VEGF-cTcR clone 2 cells were incubated with increasing amounts of human KDR-Fc (R & D Systems), washed twice with PBS, and stained with anti-human IgG₁-FITC antibody. For analysis of transduced CTLs, cells were incubated successively with anti-human c-Myc antibody, biotinylated anti-mouse IgG, and streptavidin-PE or only with anti-mouse CD8 antibody conjugated to FITC (Pharmingen).

### In Vitro Cytotoxicity Assays

Non-transduced B16.F10 cells or B16.F10 cells that were transduced with a retroviral construct encoding full-length Flk-1 were labeled with Cr⁵¹-sodium (NEN), washed with PBS, and incubated with varying amounts of primary CTLs (5-days post-transduction) in 96-well dishes for 8 hours. MILE cells were seeded onto 12-well dishes at a density of 1.5×10⁵ cells/well in endothelial cell medium. On the following day, the cells were overlaid with varying amounts of CTLs (4 day post-transduction) in TCGM and incubated for 5 hours. Cell-free supernatants were harvested and analyzed in a scintillation counter (B16.F10 cells) or by using a standard dehydrogenase (LDH) cytotoxicity kit (MILE cells) (Promega). In some experiments, MILE cells were pre-incubated with 10 μg/ml of either anti-Flk-1 antibodies or isotype-control antibodies (Pharmingen). Maximal release was calculated after incubating target cells in 1% Triton X-100.

### Treatment Of Mice With Genetically Modified T Cells

On day 0, tumor cells were implanted into the subcutaneous space on the right flank of recipient mice. On the days of treatment, CTLs (4 to 7 post-transduction) were harvested, washed, resuspended in cold PBS, and injected in a volume of 300 μl into the retro-orbital venous plexus. Mice were treated daily with 25,000 units of human recombinant interleukin-2 (IL-2) (Chiron) in 0.5 ml of PBS via intraperitoneal injection. TNP-470 (TAP Holdings Inc., Deerfield, Ill.) (30 mg/kg) was injected subcutaneously (into a site remote from the tumor) every other day in 0.3 ml PBS starting with the first day of CTL therapy. Starting volumes of tumors ranged from 40 to 80 mm^{3.} All mice were sacrificed when control mice reached a mean tumor volume of 2,000 mm³ or had extensive tumor ulceration.

Results

Generation of CD8 Lymphocytes Targeted To VEGF Receptors.

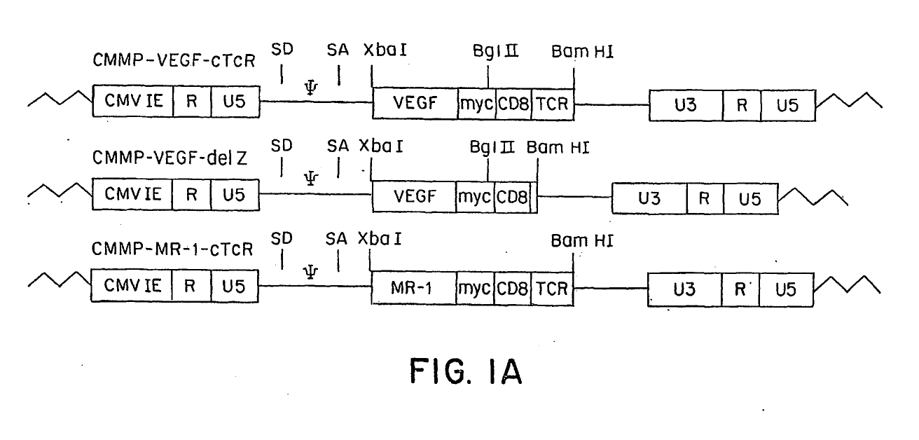

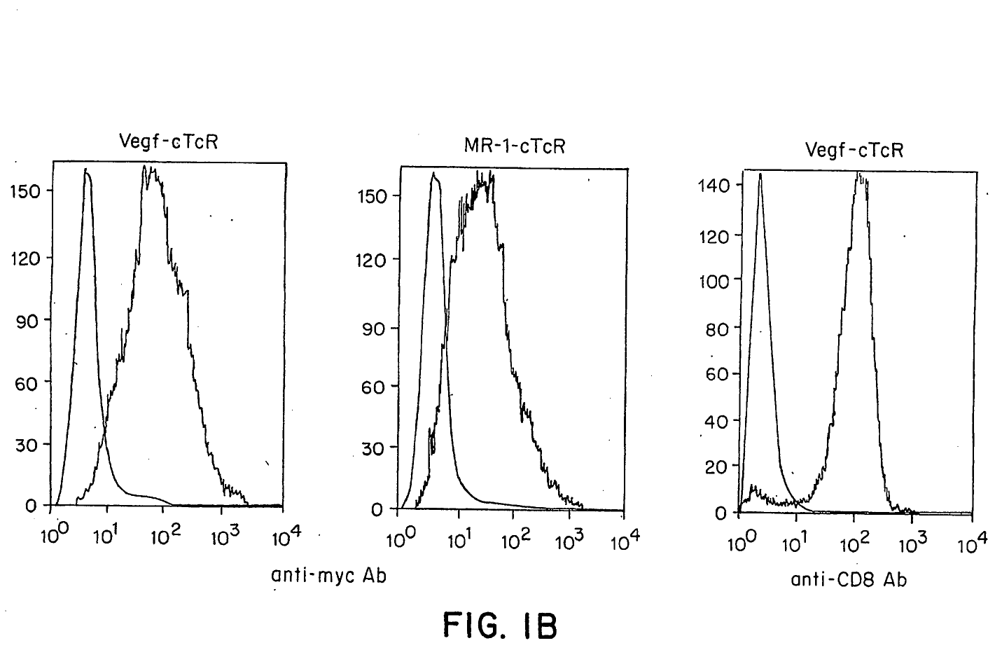

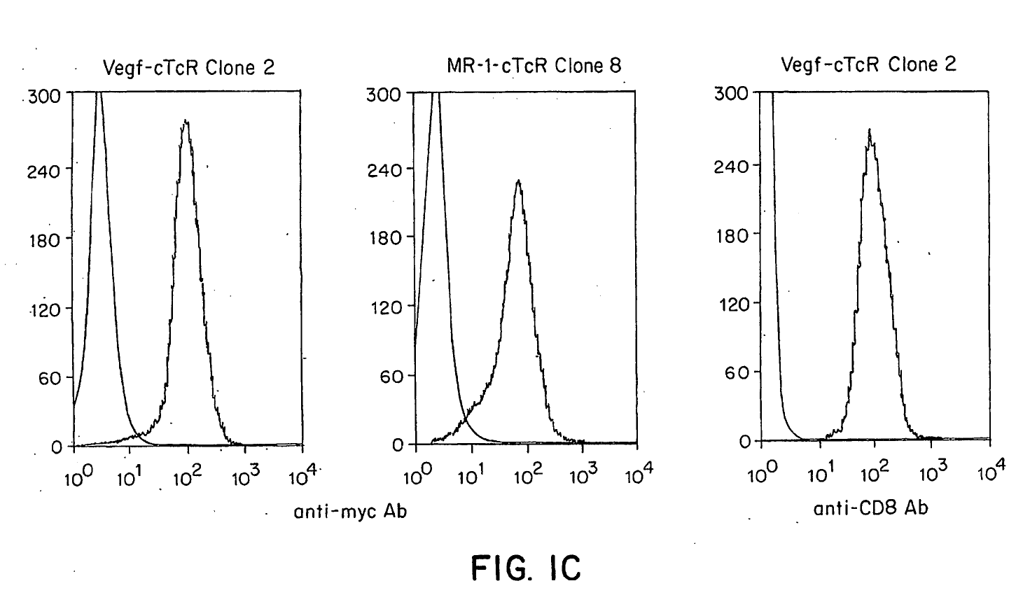

In a first step towards the generation of T lymphocytes possessing a killing specificity for VEGF receptors (VEGFRs), cDNA sequences derived from several sources were assembled to encode a chimeric T cell receptor (termed VEGF-cTcR) composed of the entire coding region of VEGF-165 (Claffey, K. P., J. Biol. Chem., 267:16317-16322 (1992)), followed by a human c-Myc epitope, a CD8α hinge region, and the transmembrane and signal transducing domain of the ζ chain of the murine CD3-T cell receptor complex (Moritz, D. et al., Proc. Natl. Acad. Sci. USA, 91:4318-4322 (1994)) (FIG. 1A). A second related chimeric T cell receptor gene, termed VEGF-cTcR del Z, which encoded a truncated form of the T cell receptor lacking the C-terminal 100 amino acids of the wild type VEGF-cTcR, was also constructed. Lastly, a chimeric T-cell receptor (MR-1-cTcR) gene which replaced the VEGF coding sequences present in VEGF-cTcR with sequences encoding a single chain monoclonal antibody directed against the antigen, EGFRvIII (Wikstrand, C. J. et al., Cancer Res., 55:3140-3148 (1995)), was also constructed. EGRFvIII is an epidermal growth factor receptor variant that is expressed on several solid tumor types but is not present in normal mice (Wikstrand, C. J. et al., Cancer Res., 55:3140-3148 (1995)).

Each of the chimeric T cell receptor genes were inserted into the retroviral vector CMMP (Klein, C. et al., J. Exp. Med., 191:1699-1708 (2000)) and high-titer recombinant virus encoding each of the gene products was produced. To generate primary murine CD8⁺ lymphocytes expressing the different chimeric T cell receptor genes, a transduction protocol was developed which involved ex vivo preactivation of CTLs, co-incubation of concentrated retroviral stock with activated CTLs on ice, and finally incubation of the CTL retrovirus suspension at 37° C. in the presence of polybrene and IL-2, as described above. Transduced cells were then maintained in the presence of T cell activators (anti-CD3 and anti-CD28 antibodies) for an additional 3 days following retroviral transduction. After infection of primary lymphocytes with either CMMP VEGF-cTcR or CMMP MR-1-cTcR viruses, over 90% of the cells efficiently expressed the relevant transgene, as determined by FACS analysis, using antibody directed to the Myc epitope present in both transgenes (FIG. 1B). Transduction of primary T cells with the VEGF-cTCR-delZ also led to efficient cell surface expression. Ninety-five percent of the transduced cells were CD8-positive 3 days post-retroviral transduction (FIG. 1B), and after day 5 of culture over 98% of cells were CD8-positive. After transduction, cTcR expression directly correlated with T cell activation status as determined by the expression of the high affinity IL-2 receptor, CD25, and cell surface expression of the chimeric receptors was maintained for at least 8 days post-transduction. Although this efficiency of gene transfer to primary lymphocytes has been previously reported by others (Altenschmidt, U. et al., J. Immunol., 159:5509-5515 (1997); Hwu, P. et al., Cancer Res., 55:3369-3373 (1995); Cost, G. L. et al., J. Immunol., 164:3581-3590 (2000); Darcy, P. K. et al, J. Immunol., 164:3705-3712 (2000); and Hagani, A. B. et al., J. Gene Med., 1:341-351 (1999)), the transduction protocol presented here is of particular interest in that it does not require either cocultivation with viral producer cells, multiple viral supernatant exposures, or antibiotic selection.

In addition to the transduction of primary murine CD8⁺ lymphocytes, the CMMP VEGF-cTcR and CMMP MR-1-cTcR viruses were also used to transduce CL96 cells, a murine CTL line (Marcucci, F. et al., Nature, 291:79-81 (1981)), and two stable cell clones, VEGF-cTcR clone 2 and MR-1-cTcR clone 8, were isolated and expanded. As shown in FIG. 1C, these clones demonstrated efficient transgene expression, even after several months in culture. Southern blot analysis of both transduced primary lymphocytes and CL96 cells indicated that the transduced CTLs contained approximately one copy of transgene per cell.

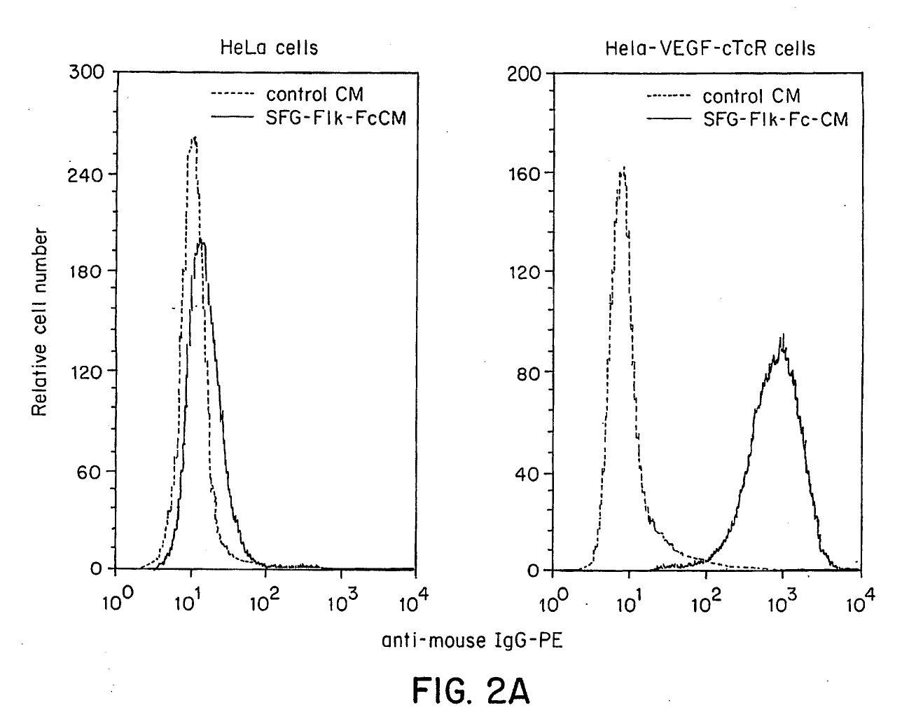

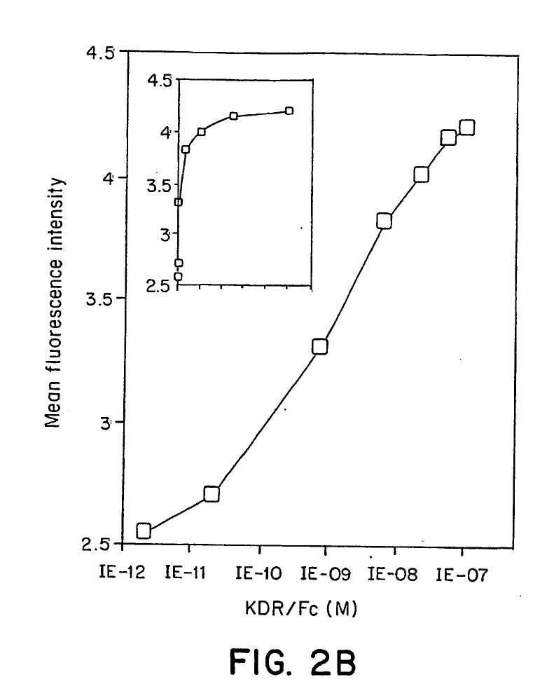

To assess whether the VEFG-cTcR encoded by the CMMP vector was capable of recognizing Flk-1, a FACS-based assay was used to directly measure the binding of a soluble form of Flk-1 (Flk-Fc) to CMMP VEGF-cTcR transduced cells. As shown in FIG. 2A, soluble Flk-Fc efficiently bound to HeLa cells expressing VEGF-cTcR but not to non-transduced HeLa cells. To facilitate the repeated measurements of the affinity of binding of Flk-Fc to cell surface VEGF-cTcR, C196 cells expressing the VEGF-cTcR gene (VEGF-cTcR clone 2 cells), rather than transduced primary cells, were used next in a binding assay involving incubation of the cells with varying amounts of purified KDR-Fc followed by the subsequent addition of a FITC-labeled secondary antibody as described above. FACS analysis of the mean fluorescence of bound secondary antibody indicated that a half-maximnal shift in mean fluorescence was achieved in the presence of 2 nM KDR-Fc (FIG. 2B). This binding affinity is comparable to that reported for the association of native VEFG and Flk-1 (0.1-0.5 nM) (Millauer, B. et al, Cell, 72:835-846 (1993); and Quinn, T. P. et al., Proc. Natl. Acad. Sci. USA, 90:7533-7537 (1993)). This affinity is noteworthy in light of the use of human rather than murine VEGFR2 in the binding assay (because of the commercial availability of KDR-Fc) and in light of previous studies which suggested that VEGF normally binds to Flk-1 as a homodimer in a head-to-tail configuration (Weismann, C. F. et al, Cell, 91:695-704 (1997)). As expected, binding of KDR-Fc to the VEGF-cTcR clone 2 cells was inhibited in a dose-dependent manner by VEGF, and there was no appreciable binding of soluble KDR-Fc to a CTL clone stably expressing MR-1-cTcR (MR-1 -cTcR clone 8 cells).

VEGF-cTcR CTLs Specifically Lyse Flk-1 Expressing Cells In vitro.

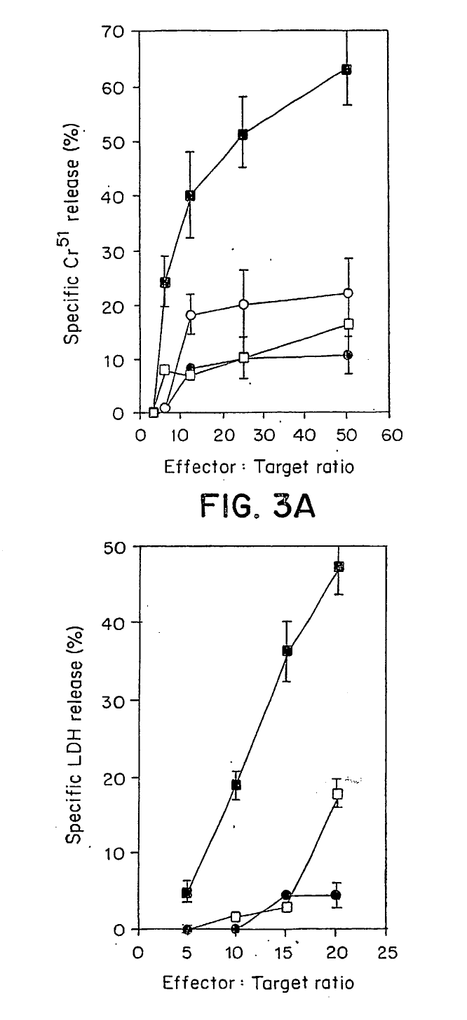

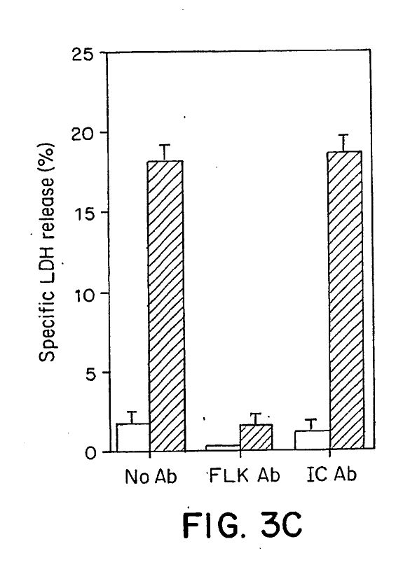

To determine whether primary CTLs transduced with the VEGF-cTcR construct could recognize and kill syngeneic cells expressing Flk-1, CTLs were incubated with either B16.F10 melanoma cells (which do not express Flk-1) or B16.F10 cells genetically modified to express Flk-1. In a standard in vitro cytotoxicity assay, CTLs transduced with the VEGF-cTcR construct, but not the MR-1-cTcR construct, specifically and efficiently lysed only the Flk-1 expressing B16.F10 cells (FIG. 3A). Moreover, there was no difference in non-specific cell death between the VEGF-cTcR CTLs and MR-1-CTLs when parental B16.F10 cells were used as cellular targets (FIG. 3A).

To assess whether VEGF-cTcR CTLs could lyse syngeneic, activated endothelial cells that naturally express Flk-1, the CTLs were incubated with MILE cells (Arbiser, J. L. et al., Proc. Natl. Acad. Sci. USA, 94:861-866 (1997)). As shown in FIG. 3B, CTLs transduced by the VEGF-cTcR construct, but not by the MR-1-cTcR construct, demonstrated specific and efficient in vitro cytotoxicity against MILE cells in a dose-dependent manner. As expected, CTLs which express on their surface the VEGF-TcR lacking cytoplasmic signalling sequences (VEGF-cTcR del Z) showed no significant cell killing in cytotoxicity assays, except at the highest effector-to-target ratio. The specific killing observed using the MILE cells was depended on the presence of Flk-1 on the surface of the endothelial cells as pre-incubation of the cells with monoclonal antibodies directed against Flk-1 (but not isotype-matched antibodies) led to a 5-10-fold reduction in MILE cell killing by VEGF-cTcR CTLs (FIG. 3C). Again, no appreciable killing was observed using T cells expressing VEGF-cTcR del Z (open bars).

VEGF-cTcR CTLs Suppress Tumor Growth It Vivo.

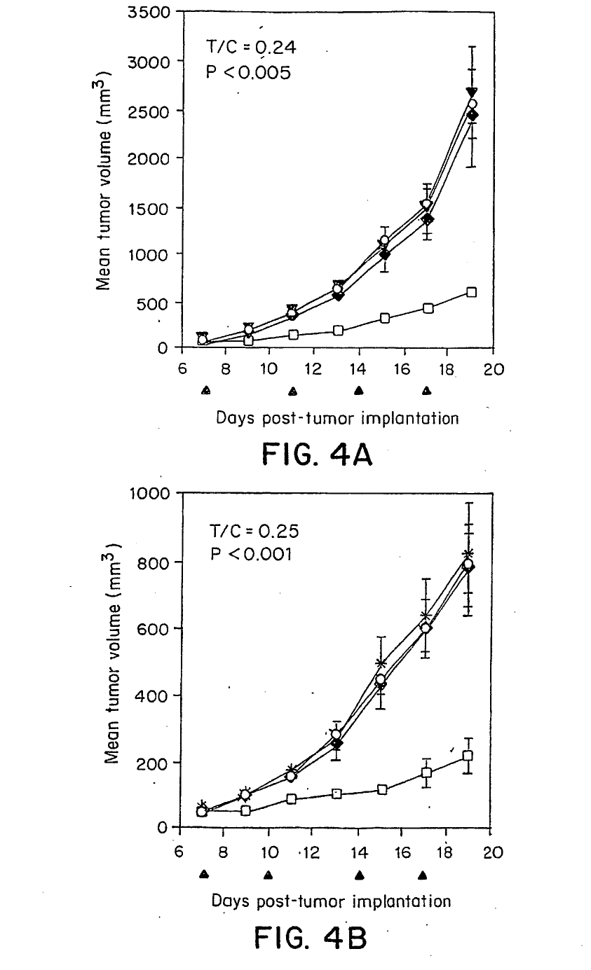

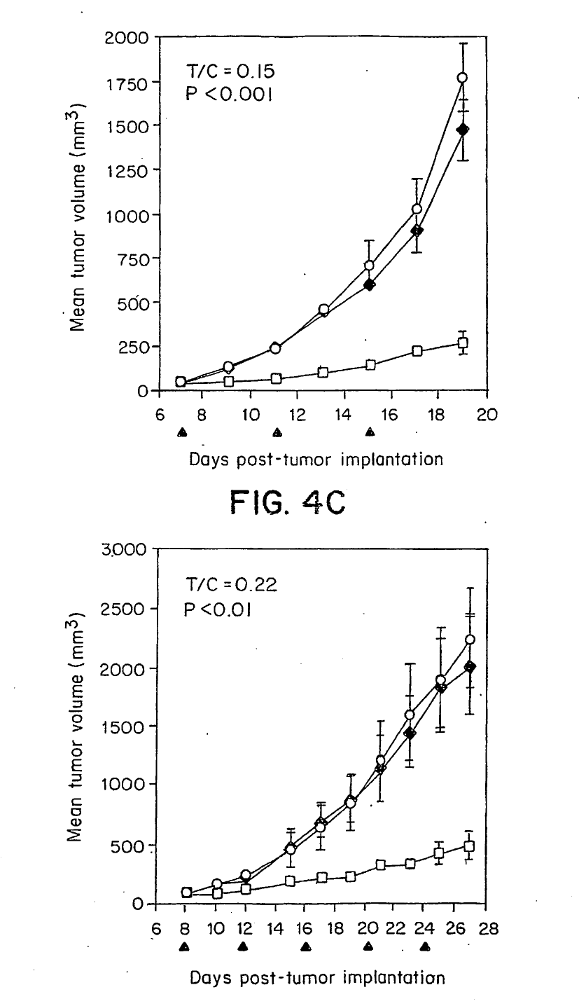

To determine whether adoptive transfer of syngeneic VEGF-cTcR CTLs could inhibit tumor growth, primary CTLs were intravenously injected into either BALB/c mice bearing CT26 murine colon adenocarcinomas or C57BL/6 mice bearing B16.F10 murine melanomas. Cytotoxic T-lymphocytes transduced with the VEGF-cTcR construct, but not the MR-1-construct, inhibited the growth of CT26 and B16.F10 tumors by 76% (FIG. 4A) and 75% (FIG. 4B), respectively, as determined by the ratio of the size of the tumors in the treated mice to that of the control mice (T/C ratio measured at day 19 post implantation). Signaling through the zeta chain of VEGF-cTcR was required for anti-tumor activity, because CTLs expessing VEGF-cTcR del Z had no impact on tumor growth (FIG. 4B). In all experiments, exogenous IL-2 was administered daily (Cheever, M. A. et al., Immunol. Rev., 157:177-194 (1997)) beginning with the first day of CTL therapy. Although IL-2 had no independent anti-tumor effect (FIG. 4A), its co-administration with the CTLs was essential to achieve significant therapeutic effect (FIG. 4B) (Hekele, A. et al., Int. J. Cancer, 68:232-238 (1996))). To achieve the observed level of inhibition, repeated administrations of chimeric T cells were required at days 7, 11, 14 and 17, and tumor growth resumed after cessation of the injection of cells and cytokine.

To determine whether VEFG-cTcR expressing C196 cells possessed anti-tumor activity after adoptive transfer comparable to transduced primary cells, a second series of studies was conducted. To enable the evaluation of the activity of the chimeric T cell receptor against both murine and human tumors, tumor bearing nude mice were used. As shown in FIGS. 4C and 4D, VEGF-cTcR clone 2 cells suppressed the growth of B16.F10 melanomas by 85% (FIG. 4C) and LS174T human colon adenocarcinomas by 78% (panel D), while the adoptive transfer of control MR-1-cTcR clone 8 cells led to no significant anti-tumor effects (FIGS. 4C and 4D).

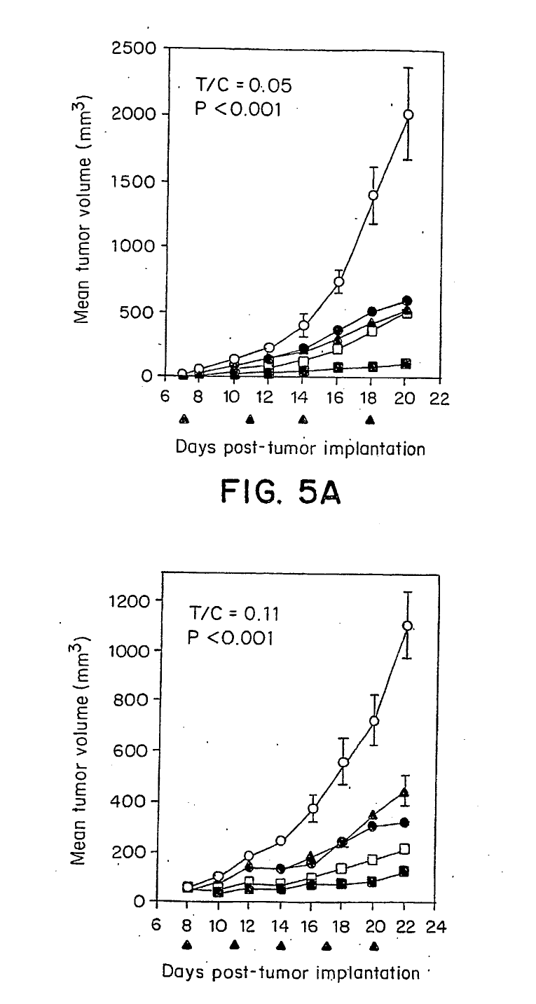

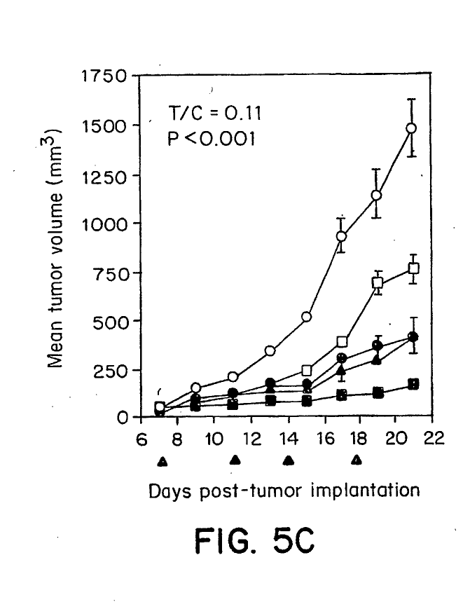

Lastly, to determine whether the anti-tumor efficacy of VEGF-cTcR primary CTLs could be enhanced by the addition of a conventional angiogenesis inhibitor, immunocompetent tumor-bearing mice were treated with a combination of the CTLs and a fumagillin analog, TNP-470 in three separate pre-existing tumor models (FIGS. 5A to 5C). When TNP-470 was combined with the VEGF-cTcR CTLs, growth of CT26 adenocarcinomas, B16.F10 melanomas and T241 fibrosarcomas was inhibited by 95%, 89%, and 90%, respectively. In comparison, treatment with TNP-470 alone suppressed the growth by 70%, 71%, and 73%, respectively, while those treated with the VEGF-cTcR CTLs alone were inhibited by 74%, 81%, and 49%, respectively (FIGS. 5A to 5C). In contrast, the addition of non-specific CTLs to TNP-470 therapy was no more efficacious than treatment with TNP-470 alone (FIGS. 5A to 5C). Mice treated with TNP-470 alone displayed a weight loss of up to 10% of total body weight and occasionally had mild skin breakdown at the TNP-470 injection site, but no further toxicity was noted in mice that concomitantly received CTL therapy.

The teachings of all the articles, patents and patent applications cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A modified T lymphocyte comprising a chimeric T cell receptor reactive with two or more different cell surface angiogenic markers.

2. The modified T lymphocyte of claim 1 wherein one of the angiogenic markers is a vascular endothelial growth factor 2 receptor.

3. The modified T lymphocyte of claim 2 wherein the vascular endothelial growth factor 2 receptor is KDR.

4. The modified T lymphocyte of claim 2 wherein the vascular endothelial growth factor 2 receptor is Flk-1.

5. A retroviral vector encoding a chimeric T cell receptor reactive with two or more different cell surface angiogenic markers comprising:
a) a nucleotide sequence coding for a secreted VEGF2 molecule or a binding portion thereof, wherein said VEGF2 molecule or binding portion thereof is capable of binding two or more different cell surface angiogenic markers;
b) an intracellular signal transduction sequence; and
c) a hinge sequence located between the nucleotide of a) and the intracellular signal transduction sequence of b).

6. The retroviral vector of claim 5 wherein one of the angiogenic markers is a vascular endothelial growth factor 2 receptor.

7. The retroviral vector of claim 6 wherein the vascular endothelial growth factor 2 receptor is KDR or Flk-1.

8. The retroviral vector of claim 5 wherein the signal transduction sequence is selected from the group consisting of: a nucleotide sequence of the zeta chain of the CD3-T cell receptor complex, a nucleotide sequence of the gamma chain of the Fcε receptor I complex and a nucleotide sequence of the gamma chain of the Fcγ receptor IIII complex.

9. The retroviral vector of claim 5 further comprising a detectable epitope sequence for detecting said chimeric T cell receptor when expressed on transduced T lymphocytes.

10. A chimeric T cell receptor reactive with two or more different cell surface angiogenic markers comprising:
a) an extracellular binding domain capable of binding two or more different cell surface angiogenic markers, said binding domain encoded by VEGF2 coding sequences;
b) an intracellular signal-transducing domain; and
c) a hinge region located between the binding domain of a) and the signal-transducing domain of b).

11. The chimeric T cell receptor of claim 10 wherein one of the angiogenic markers is a vascular endothelial growth factor 2 receptor.

12. The chimeric T cell receptor of claim 11 wherein the vascular endothelial growth factor 2 receptor is KDR or Flk-1.

13. The chimeric T cell receptor of claim 10 wherein the signal transducing domain is selected from the group consisting of: a domain of the zeta chain of the CD3-T cell receptor complex, a domain of the gamma chain of the Fcε receptor I complex and a domain of the gamma chain of the Fcγ receptor IIII complex.

14. The chimeric T cell receptor of claim 10 further comprising a detectable epitope for detecting said chimeric T cell receptor when expressed on transduced T lymphocytes.

15. A packaging cell line for producing a retroviral vector encoding a chimeric T cell receptor reactive with two or more different cell surface angiogenic markers comprising:
a) a mammalian cell;
b) a first retroviral nucleotide sequence in the cell which comprises a coding sequence for viral gagpol proteins;
c) a second retroviral nucleotide sequence in the cell which comprises a coding sequence for a heterologous envelope protein; and
d) a third retroviral nucleotide sequence in the cell which comprises a coding sequence for a chimeric T cell receptor reactive with two or more different cell surface angiogenic markers.

16. A method of producing a packaging cell line for producing a retroviral vector encoding a chimeric T cell receptor reactive with two or more different cell surface angiogenic markers, comprising co-transfecting mammalian host cells with:
a) a first plasmid comprising a DNA sequence which encodes viral gagpol proteins;
b) a second plasmid comprising a DNA sequence which encodes a heterologous envelope protein; and
c) a third plasmid comprising a DNA sequence which encodes a chimeric T cell receptor reactive with two or more different cell surface angiogenic markers.

17. A method of producing modified T lymphocytes that express a chimeric T cell receptor reactive with two or more different cell surface angiogenic markers, comprising:
a) pre-activating T lymphocytes with anti-CD28 and anti-CD3 antibodies;
b) co-incubating the activated T lymphocytes from step a) on ice with a retroviral vector encoding said chimeric T cell receptor reactive with two or more different cell surface angiogenic markers, thereby producing a suspension of T lymphocytes and retrovirus;
c) incubating the suspension from step b) at 37° C. in the presence of IL-2 and polybrene,
whereby modified T lymphocytes expressing said chimeric receptor reactive with two or more different cell surface angiogenic markers are produced.

18. The method of claim 17 wherein one of the angiogenic markers is a vascular endothelial growth factor 2 receptor.

19. The method of claim 18 wherein the vascular endothelial growth factor 2 receptor is KDR or Flk-1.

20. A method of suppressing or inhibiting tumor growth in a patient having a tumor comprising:
a) transducing T lymphocytes obtained from said patient with a retroviral vector encoding a chimeric T cell receptor reactive with two or more different cell surface angiogenic markers, thereby producing modified T lymphocytes expressing said chimeric T cell receptor reactive with two or more different cell surface angiogenic markers; and
b) administering to said patient modified T lymphocytes produced in step a) and an effective amount of IL-2.

21. The method of claim 20 wherein one of the angiogenic markers is a vascular endothelial growth factor 2 receptor.

22. The method of claim 21 wherein the vascular endothelial growth factor 2 receptor is KDR.

23. The method of claim 20 further comprising administering to said patient an angiogenesis inhibitor.

24. The method of claim 23 wherein said angiogenesis inhibitor is a fumagillin analog.
